(19) United States
(12) Patent Application Publication
(10) Pub. No.: US 2024/0339220 A1
(43) Pub. Date: Oct. 10, 2024

(54) LONGITUDINAL PREDICTIVE MODEL FOR PREDICTING ADVERSE GESTATIONAL OUTCOMES

(71) Applicants: NX PRENATAL INC., Louisville, KY (US); The Brigham and Women’s Hospital, Inc., Boston, MA (US)

(72) Inventors: Thomas F. MCELRATH, Dover, MA (US); Kevin P. ROSENBLATT, Bellaire, TX (US); Brian D. BROHMAN, Louisville, KY (US)

(21) Appl. No.: 18/579,348

(22) PCT Filed: Jul. 13, 2022
(86) PCT No.: PCT/US2022/037022

§ 371 Date: Jan. 13, 2024

Related U.S. Application Data
(60) Provisional application No. 63/222,360, filed on Jul. 15, 2021.

(51) Int. Cl. G16H 50/30; G16H 10/60; G16H 50/20; G16H 50/70

(52) U.S. Cl. CPC G16H 50/30; G16H 10/60; G16H 50/20; G16H 50/70

(57) ABSTRACT

A method for developing a longitudinal predictive model for adverse gestational outcomes comprises receiving pregnancy datasets from discrete data sources for a plurality of subjects having a plurality of gestational outcomes, independently analyzing the discrete datasets to identify biomarkers for the gestational outcomes, combining the biomarkers into a single meta-dataset, and analyzing the meta-dataset to produce a model for the gestational outcome. Models can be generated for a plurality of different timepoints in pregnancy. A pregnant subject can be tracked into one of a plurality of treatment tracks based on predicted risk of an adverse gestational outcome after each episode of testing.

### CROSS-REFERENCE TO RELATED APPLICATIONS

This application claims priority to U.S. Patent Application No. 63/222,360, filed on Jul. 15, 2021, the contents of which is incorporated herein by reference in its entirety.

### BACKGROUND

Preterm birth is the delivery of a baby fewer than 37 weeks of gestational age. It results in significant morbidity and mortality to both mother and infant, and is the most common cause of infant death worldwide. Currently, there is no complete treatment for preterm birth. The condition is managed through a combination of medications (e.g., progestogens, low-dose aspirin, steroids, antibiotics, tocolytics), mode of delivery, and intensified neonatal care regimens. Methods of predicting and diminishing incidence of preterm birth would advance obstetrics and gynecology

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1A shows a two-step method of developing a diagnostic model. It includes independent analysis of a plurality of “level 1” datasets to identify biomarkers, combination of the biomarkers into a single “level 2” meta-dataset, and analysis of the meta-dataset to produce the ultimate predictive model.

FIG. 1B is a flow chart illustrating the method of developing a diagnostic model, in accordance with some embodiments.

FIG. 2 shows an exemplary first-level analysis of data from discrete data sources.

FIG. 3 shows an exemplary second-level analysis of data derived from first-level analysis.

FIG. 4 shows a timeline of pregnancy and exemplary sources of clinical data and microparticle molecular data obtainable at different points during pregnancy. Sources include maternal data inputs, conception data inputs, liquid biopsy data via exosomes, NIPT fetal genomic data and clinical inputs. Exemplary model training outcomes (categories of prediction) are also shown.

FIG. 5 shows exemplary therapeutic tracks for subjects classified into high, medium and low preterm birth risk states, at each of three different time points at which predictions are made.

FIG. 6 shows an exemplary system for predicting adverse gestational outcome.

### SUMMARY

Methods for longitudinal tracking of subjects at risk of preterm birth include developing diagnostic models that incorporate several different types of data at a plurality of different time points during pregnancy. In the generation of diagnostic models, subjects are categorized in terms of the gestational outcome of their pregnancy. The datasets used in both model creation and subsequent tracking of individual subjects can include clinical data, microparticle-derived molecular data and comparand output data (comparison of data from datasets taken at different time points. Clinical data can include maternal inputs that can be determined before pregnancy, conception data, and pregnancy clinical data. Microparticle-derived molecular data can include data from exosomes taken from pregnant subjects, for example during first and second trimesters. Molecular data can be obtained from NIPT genomic data. Comparand output data can include data showing differences between microparticle data collected at different time points in pregnancy. In the application of the diagnostic, at a plurality of times during pregnancy, pregnant subjects can be assessed for risk of an adverse gestational outcome and tracked into different treatment tracks, based on level of risk of an adverse gestational outcome.

In one aspect, a method of generating a model that infers a gestational outcome in a subject in the second trimester of pregnancy is described herein. The method can comprise receiving at a controller a plurality of datasets comprising data on each of a plurality of subjects. The datasets can comprise: i) one or more datasets comprising measures of clinical data, ii) a dataset comprising measures of first trimester microparticle data, iii) a dataset comprising measures of second trimester microparticle data, and iv) a comparand output dataset comparing data from first trimester data to second trimester data. Each dataset can include a gestational outcome identifier for each subject. The method can also comprise performing an analysis on each of the datasets by computer. The analyses can identify one or a plurality of dataset features from each dataset that infer a gestational outcome in subject. The method can also comprise computing via a controller a meta-dataset that includes for each subject measures of a plurality of the identified features from each of the datasets and the gestational outcome identifier. The method can also include generating via the controller and based on the analysis of the meta-dataset a model that infers a gestational outcome for a subject from the identified features.

In some variations, the gestational outcome can be one or more of: initiation of preterm labor, spontaneous preterm birth, birth weight, neonatal intensive care unit admission/length of stay, Hassan score, necrotizing enterocolitis (NEC) and rehospitalization within one year. In some variations, the model can infer an adverse gestational outcome. The gestational outcome inferred can be a risk score of the gestational outcome.

In some variations, the plurality of subjects is at least 25, at least 50, at least 200, at least 500, at least 1000 or at least 10,000. The clinical data can comprise data from one or more groups consisting of (i) pre-pregnancy maternal data, (ii) conception data, (iii) pregnancy maternal data, (iv) NIPT fetal genomic data, and (v) radiographic data. The clinical data can comprise a plurality of separate datasets. The datasets can comprise clinical data comprising a plurality of datasets. Each dataset can comprise data received from a plurality of different timepoints in pregnancy (e.g., one or more timepoints in each of two or more different trimesters).

In some variations, the pre-pregnancy maternal data comprises one or more of: (1) a social determinants of health, (2) prior episode of preterm birth, (3) prior episode of preeclampsia, (4) prior stillbirth, (5) prior miscarriage, (6) presence or absence of a chronic health condition, (7) a prior gynecological complication, (8) race/ethnicity, (9) smoking, (10) drug use, and (11) body mass index. In some variations, the pre-pregnancy maternal data comprises one or more social determinants of health selected from access to healthcare; healthcare insurance status; social status; social support networks; educational attainment; employment/working conditions; social environments; physical environments; community exposure to pollutants; personal health practices and coping skills; healthy child development; and culture.

In some variations, the conception data comprises one or more of in vitro fertilization status, artificial conception status, and time interval from prior pregnancy. The pregnancy maternal data comprises one or more of: physician clinical observations, results of physical examinations, blood and/or urine testing values, ultrasound assessments, presence or absence of bleeding, blood pressure data, presence or absence of gestational diabetes, and symptoms of preterm labor. The NIPT fetal genomic data comprise one or more of fetal sex and presence or absence of fetal genetic abnormality

In some variations, the microparticle-derived molecular data comprises liquid biopsy data. The liquid biopsy data comprises exosome-derived data. The microparticle data regarding fetal development status comprises one or more biomarkers of management of oxidative stress, proper nutrient supply, metabolism of cholesterol, wound healing, and management of inflammatory processes. The microparticle data regarding immune adaptation status comprises biomarkers involved in the regulation of the complement cascade. The microparticle data regarding utero-placental accommodation status comprises one or more biomarkers related to embryo implantation, placentation, cytotrophoblastic invasion of the maternal decidua, abnormal placental development, angiogenesis and spiral artery remodeling to a low resistance phenotype.

In some variations, the identified features of the first and second trimester microparticle datasets are not identical. In some variations, the gestational outcome identifier comprises one or more of the following indicators of preterm labor initiation: progesterone withdrawal, PR-A/PR-B ratio switch, cervical shortening via trans-abdominal or trans-vaginal ultrasound, and fetal fibronectin in cervical-vaginal fluid.

In some variations, the comparand output dataset compares the data by linear, logarithmic or normalized differences. In some variations, providing the dataset of measures of microparticle data comprises (I) preparing a microparticle-enriched fraction from a blood sample from the pregnant subject, and (II) determining a quantitative measure of microparticle-associated proteins in the fraction. In some variations, the first trimester data is collected between 10 and 12 weeks of pregnancy. In some variations, the second trimester data is collected between 24 and 26 weeks of pregnancy.

In some variations, the liquid biopsy data can comprise protein data. The blood sample can be a serum sample or a plasma sample. The microparticle-enriched fraction can be prepared using size-exclusion chromatography. The size-exclusion chromatography can comprise elution with distilled, deionized H₂O. In some variations, the size-exclusion chromatography can be performed with an agarose solid phase and an aqueous liquid phase. In some variations, the preparing step can further comprise using ultrafiltration or reverse-phase chromatography. In some variations, the preparing step can further comprise denaturation using urea, reduction using dithiothreitol, alkylation using iodoacetamine, and digestion using trypsin.

In some variations, the analyses can comprise an analysis independently selected from: regression analysis (e.g., simple regression, multiple regression, linear regression, non-linear regression, logistic regression, polynomial regression. stepwise regression, ridge regression, lasso regression, elasticnet regression) correlational, Pearson correlation, Spearman correlation, chi-square, comparison of means (e.g., paired T-test, independent T-test, ANOVA), and non-parametric analysis (e.g., Wilcoxon rank-sum test, Wilcoxon sign-rank test, sign test), as CART—classification and regression trees), artificial neural networks such as back propagation networks, discriminant analyses (e.g., Bayesian classifier or Fischer analysis), logistic classifiers, and support vector classifiers (e.g., support vector machines).

In one aspect, a method for inferring a gestational outcome in a subject during second trimester is described herein. The method can comprise receiving data from a pregnant subject. The data can comprise measures of one or a plurality of features identified from each of: i) one or more datasets comprising measures of clinical data, ii) a dataset comprising measures of first trimester microparticle data, iii) a dataset comprising measures of second trimester microparticle data, and iv) a comparand output dataset that includes data from first trimester data compared to second trimester data. The first trimester microparticle data and the second trimester microparticle data can be received by: I) providing a microparticle-enriched blood sample from a pregnant female subject, and II) determining a measure of each of a plurality of proteins from the sample. The method can also comprise producing a meta-dataset that comprises measures of the plurality of features, and executing by computer a model that infers a risk of an adverse gestational outcome in the subject from measures of the one or more features in the meta-dataset.

In some variations, the model can be created by the method of generating a model that infers a gestational outcome in a subject in the second trimester of pregnancy as described herein.

In one aspect, a method of treating a pregnant subject is described herein. The method can comprise (I) during pregnancy: inferring a gestational outcome in a pregnant subject by executing a model on a first meta-dataset that includes measures of a plurality of diagnostic features for the subject, and tracking the subject into one of three treatment tracks selected from: traditional prenatal care, prenatal care with telemedicine and enhanced at risk care based on an inference of low, average, or high risk of an adverse gestational outcome. The measures of the plurality of diagnostic features for the subject can be from one or more first datasets comprising measures of clinical data. The clinical data can comprise maternal data inputs and/or conception data inputs. The method can also comprise (II) during the first trimester of pregnancy, and after (I): inferring a gestational outcome in the subject by executing a model on a second meta-dataset that includes measures of a plurality of diagnostic features for the subject. These measures can be from: one or more first datasets comprising measures of clinical data, wherein the clinical data comprises maternal data inputs and/or conception data inputs, a dataset comprising measures of first trimester microparticle data, optionally, a first trimester clinical dataset comprising measures of clinical data collected during the first trimester. The method can also comprise (II) during the first trimester of pregnancy, and after (I) tracking the subject into one of three treatment tracks selected from: traditional prenatal care, prenatal care with telemedicine and enhanced at risk care) based on an inference of low, average, or high risk of an adverse gestational outcome. The method can also comprise (III) during the second trimester of pregnancy: inferring a gestational outcome in the subject by executing a model on a third meta-dataset that includes measures of a plurality of diagnostic features for the subject. These measures can be from: i) one or more first datasets comprising measures of clinical data, wherein the clinical data comprises maternal data inputs and/or conception data inputs, ii) a dataset comprising measures of first trimester microparticle data, iii) optionally, a first trimester clinical dataset comprising measures of clinical data collected during the first trimester, iv) a dataset comprising measures of second trimester microparticle data, v) a comparand output dataset comparing data from first trimester microparticle data to second trimester microparticle data, vi) optionally, one or more third clinical datasets comprising measures of clinical data collected during the second trimester. The method can further comprise tracking the subject into one of three treatment tracks selected from: traditional prenatal care, prenatal care with telemedicine, and enhanced at-risk care based on an inference of low, average, or high risk of an adverse gestational outcome.

In some variations, a tracking decision can be performed by a model. In some variations, enhanced at-risk care can comprise one or more of: 1. Referral to Preterm Birth Prevention Clinic, 2. Referral to Maternal Fetal Medicine specialist, 3. Education on signs/symptoms of preterm labor, 4. Evaluation of medical (i.e. progestogen supplementation, low-dose aspirin) or surgical (i.e. cervical cerclage) options, 5. Modification of behaviors, lifestyle and diet to support a healthy birth outcome, 6. Increased office visits and modified content of office visits, 7. Increased surveillance via ultrasound and cervical length measurements, and 8. Preparation for acute-stage events (i.e. planning for NICU access, education on medicines that can be given upon initiation of preterm labor to extend gestation, mature the baby's lungs, and provide neuroprotective agents for the baby's brain development).

In one aspect, a method for treating a pregnant subject at high risk of an adverse gestational outcome is described herein. The method can comprise receiving a meta-dataset that includes for the subject, measures of a plurality of diagnostic features from: i) one or more first datasets comprising measures of clinical data, wherein the clinical data comprises maternal data inputs and/or conception data inputs, and ii) a dataset comprising measures of first trimester microparticle data, iii) optionally, a first trimester clinical dataset comprising measures of clinical data collected during the first trimester, and iv) a dataset comprising measures of second trimester microparticle data, v) an output dataset comparing data from first trimester microparticle data to second trimester microparticle data, vi) optionally, one or more third clinical datasets comprising measures of clinical data collected during the second trimester. The method can further comprise (B) executing a model that predicts an adverse gestational outcome in the subject based on measure of the diagnostic features by computer, and (C) tracking the subject into one of three treatment tracks selected from: traditional prenatal care, prenatal care with telemedicine and enhanced at risk care based on an inference of low, average, or high risk of an adverse gestational outcome.

In one aspect, a system for inferring risk of an adverse gestational outcome is described herein. The system can comprise (a) a controller; and (II) a database coupled to the controller. The controller can comprise: (1) a meta-dataset comprising measures of features in feature data, wherein the feature data comprises: i) one or more first datasets comprising measures of clinical data, wherein the clinical data comprises maternal data inputs and/or conception data inputs, and ii) a dataset comprising measures of first trimester microparticle data, iii) optionally, a first trimester clinical dataset comprising measures of clinical data collected during the first trimester, and iv) a dataset comprising measures of second trimester microparticle data, v) an output dataset comparing data from first trimester microparticle data to second trimester microparticle data, vi) optionally, one or more third clinical datasets comprising measures of clinical data collected during the second trimester. The memory storing a module can also comprise: (2) a classification model which, based on the measures, infers a gestational outcome in the subject, and (3) computer executable instructions for implementing the classification model on the meta-dataset.

In one aspect, a non-transitory computer readable medium comprising machine executable code, which, when executed by a processor, infers an adverse gestational outcome in a subject is described herein. The software product can infer the adverse gestational outcome by: (a) accessing a meta-dataset measures of features in feature data, wherein the feature data comprises: i) one or more first datasets comprising measures of clinical data, wherein the clinical data comprises maternal data inputs and/or conception data inputs, and ii) a dataset comprising measures of first trimester microparticle data, iii) optionally, a first trimester clinical dataset comprising measures of clinical data collected during the first trimester, and iv) a dataset comprising measures of second trimester microparticle data, v) an output dataset comparing data from first trimester microparticle data to second trimester microparticle data, vi) optionally, one or more third clinical datasets comprising measures of clinical data collected during the second trimester, and (b) executing a model on the meta-dataset set to infer an adverse gestational outcome in the subject.

In one aspect, a method of treating adverse gestational outcome in a subject is described herein. The method can comprise: (a) inferring the presence of adverse gestational outcome in a subject according to a method as described herein, and (b) administering a therapeutic intervention to the subject effective to treat the adverse gestational outcome.

In one aspect, a method for diagnosing and treating an adverse gestational outcome in a subject is described herein. The method can comprise: (a) receiving from a subject: (i) pre-pregnancy maternal data, (ii) conception data, (iii) pregnancy maternal data, (b) receiving from the subject a blood sample, (c) determining, from a microparticle-enriched portion of the blood sample, data comprising measures of protein features, (d) generating a meta-dataset for the subject based upon the data, (e) generating an inference of the adverse gestational outcome in the subject upon processing the meta-dataset with an inference model derived from a population of subjects, and (f) at an output device associated with the subject, providing a therapy to the subject for the adverse gestational outcome upon processing the inference with a therapy model designed to treat the adverse gestational outcome.

In one aspect, a method for creating a model that infers a gestational outcome in a subject in a first trimester of pregnancy is described herein. The method can comprise a) receiving into a database a plurality of datasets comprising data on each of a plurality of subjects, wherein the datasets include: i) one or more datasets comprising measures of clinical data, and ii) a dataset comprising measures of first trimester microparticle data. Each dataset can include a gestational outcome identifier for each subject. The method can further comprise b) performing an analysis on each of the datasets by a controller. The analyses identify one or a plurality of dataset features that infer a gestational outcome in subject from each dataset. The method can also comprise c) receiving into the database a meta-dataset that includes, for each subject, measures of a plurality of the identified features from each of the datasets and the gestational outcome identifier, and d) performing, by the controller, an analysis on the meta-dataset. The analysis produces a model that infers a gestational outcome for a subject from the identified features.

In one aspect, a method for creating a model that infers a gestational outcome in a subject in post-conception is described herein. The method can comprise: a) receiving into a database a plurality of datasets comprising data on each of a plurality of subjects, wherein the datasets include: i) a dataset comprising measures of pre-pregnancy maternal data, and ii) a dataset comprising measures of conception status data, and wherein each dataset includes a gestational outcome identifier for each subject. The method can also comprise b) performing, an analysis on each of the datasets by the controller. The analyses identify one or a plurality of dataset features that infer a gestational outcome in subject from each dataset. The method can also comprise c) receiving into a database a meta-dataset that includes, for each subject, measures of a plurality of the identified features from each of the datasets and the gestational outcome identifier, and d) performing, by the controller, an analysis on the meta-dataset. The analysis produces a model that infers a gestational outcome for a subject from the identified features.

### DETAILED DESCRIPTION

### I. Introduction

Provided herein are methods and systems to predict adverse gestational outcomes, such as preterm birth, and to manage the care of pregnant females at increased risk for such adverse gestational outcomes. The methods involve analyzing data of a plurality of different types and at a plurality of different times during pregnancy, and tracking pregnant females into different pregnancy care tracks/treatment tracks, based on assessed risk at different time points. Data sources can include one or more of clinical data of different types and collected at different points in pregnancy, microparticle-derived molecular data taken in the first trimester and in the second trimester, and differences between first and second trimester microparticle data. At each of a plurality of timepoints, a pregnant female can be assessed and tracked, and care tracks/treatment tracks can be changed based on changes in assessed risk. Risk assessments at each time point can include cumulative data from previous time points. Care tracks/treatment tracks can be tiered based on risk levels, such as “low risk,” “moderate risk,” and “high risk.” Predicting risk and tracking subjects can involve, first, building prediction models based on data from the various sources, and, second, executing these models on data from individual subjects.

The technology disclosed herein can predict the risk levels with improved accuracy over existing technology. For instance, the area under the receiver operating characteristic curve shows an improvement of at least 20 points over existing methodologies. Additionally, the technology described herein incorporates data into a diagnostic model (described below) in a manner such that the computational time to predict the risk levels is reduced. For example, rather than incorporating the entire first trimester microparticle data and the entire second trimester microparticle data at the second trimester point, the technology described herein incorporates a difference between the first trimester data and the second trimester data into the diagnostic model, thereby cutting down on execution time and/or runtime. Furthermore, the adaptive nature of the diagnostic model may improve prediction with each subsequent time point.

### II. Building a Longitudinal Model for Gestational Outcomes from Multiple Input Sources

Model building begins with receipt of data from a plurality of subjects whose gestational outcome is known or becomes known, and analyzing the data to find associations between elements of the data and the gestational outcome to be predicted. In some embodiments, the received data can be collected from a plurality of subjects and/or from one or more data sources.

### III. Data Receipt Processing

### A. Subjects

Subjects for prediction and treatment of gestational outcome requiring delivery in <=35 weeks gestation are pregnant human females. In some embodiments, the pregnant woman is in the first trimester (e.g., weeks 1-12 of gestation), second trimester (e.g., weeks 13-28 of gestation) or third trimester (e.g., weeks 29-37 of gestation) of pregnancy. In some embodiments, the pregnant woman is in early pregnancy (e.g., from 8, 9, 10, 11, 12, 13, 14, 15, 16, 17, 18, 19 or 20, but earlier than 21 weeks of gestation; from 20, 19, 18, 17, 16, 15, 14, 13, 12, 11, 10 or 9, but later than 8 weeks of gestation). In some embodiments, the pregnant woman is between 8-15 weeks of pregnancy, for example, 10-12 weeks, 8-12 weeks or 10-15 weeks. In some embodiments, the pregnant woman is in mid-pregnancy (e.g., from 21, 22, 23, 24, 25, 26, 27, 28, 29 or 30, but earlier than 31 weeks of gestation; from 30, 29, 28, 27, 26, 25, 24, 23, 22 or 21, but later than 20 weeks of gestation). In some embodiments, the pregnant woman is in late pregnancy (e.g., from 31, 32, 33, 34, 35, 36 or 37, but earlier than 38 weeks of gestation; from 37, 36, 35, 34, 33, 32 or 31, but later than 30 weeks of gestation). In some embodiments, the pregnant woman is in less than 17 weeks, less than 16 weeks, less than 15 weeks, less than 14 weeks or less than 13 weeks of gestation; from 20, 19, 18, 17, 16, 15, 14, 13, 12, 11, 10 or 9, but later than 8 weeks of gestation). The stage of pregnancy can be calculated from the first day of the last normal menstrual period of the pregnant subject.

Pregnant subjects of the methods described herein can belong to one or more classes including primiparous (no previous child brought to delivery, interchangeably referred to herein as nulliparous or parity=0) or multiparous (at least one previous child brought to at least 20 weeks of gestation, referred to interchangeably herein as parity >0, parity ≥1), primigravida (first pregnancy) or multigravida (more than one pregnancy).

### 1. Gestational Outcome

Subjects used in the development of models are categorized based on their gestational outcome. Gestational outcome also is an inference or prediction made of individuals to whom the models are applied.

Gestational outcome can refer to any characteristic of a pregnancy or a newborn infant. Gestational outcomes include, without limitation, initiation of preterm labor, spontaneous preterm birth, preeclampsia, miscarriage, stillbirth, birth weight, neonatal intensive care unit admission/length of stay, Hassan score, necrotizing enterocolitis (NEC) and rehospitalization within one year. Negative or undesirable gestational outcomes are referred to herein as “adverse gestational outcomes.” Inferences about gestational outcome can be categorical, or can be reflect level of risk or probability of the outcome.

In some embodiments, the pregnant human subject is asymptomatic. In some embodiments, the subject may have a risk factor of gestational outcome such as high blood pressure, protein in the urine, a family history of gestational outcome, renal or connective tissue disease, obesity, advanced maternal age, or a conception with medical assistance.

### B. Data Sources

### 1. Liquid Biopsy Data

As used herein, the term “liquid biopsy” refers to a biopsy of non-solid biological tissue, e.g., blood. Accordingly, any materials that can be recovered from blood can be the source or liquid biopsy data. This includes, without limitation, data derived from microparticles and data derived from cell-free nucleic acids (e.g., cell-free DNA).

### 2. Microparticle Data

In some embodiments, a source of data used in the methods described herein is microparticle data. The term “microparticle” refers to an extracellular microvesicle or lipid raft protein aggregate having a hydrodynamic diameter of about 50 to about 5000 nm. As such, the term microparticle encompasses exosomes (about 50 to about 100 nm), microvesicles (about 100 to about 300 nm), ectosomes (about 50 to about 1000 nm), apoptotic bodies (about 50 to about 5000 nm) and lipid-protein aggregates of the same dimensions. Microparticles typically are collected from peripheral blood of a subject, but can be sourced from other liquids or tissues.

The term “microparticle-associated protein” refers to a protein or fragment thereof that is detectable in a microparticle-enriched sample from a mammalian (e.g., human) subject. As such the term “microparticle-associated protein” is not restricted to proteins or fragments thereof that are physically associated with microparticles at the time of detection.

The term “polypeptide” as used herein refers to a polymer of amino acids. This includes oligopeptides, which typically have fewer than 10 amino acids, peptides, which typically have between about 10 and about 50 amino acids, and proteins, which include polypeptides assuming secondary, tertiary or quaternary structures. Depending on context, the term “protein” may refer to a polypeptide lacking secondary structure.

The term “about” as used herein in reference to a value refers to 90% to 110% of that value. For instance, a diameter of about 1000 nm is a diameter within the range of 900 nm to 1100 nm.

A sample for use in the methods of the present disclosure is a biological sample obtained from a pregnant subject. In certain embodiments, the sample is collected during a stage of pregnancy described in the preceding section. In some embodiments, the sample is a blood, saliva, tears, sweat, nasal secretions, urine, amniotic fluid or cervicovaginal fluid sample. In some embodiments, the sample is a blood sample, which in certain embodiments are serum or plasma. In some embodiments, the sample has been stored frozen (e.g., −20° C. or −80° C.).

Biomarkers for gestational outcome can be derived from microparticles. Microparticles can be isolated from blood (e.g., serum or plasma) or other biological samples, by size exclusion chromatography. The elution buffer can be, for example, a buffered solution such as PBS, a non-buffered solution, water, or de-ionized water. The high molecular weight fraction can be collected to obtain a microparticle-enriched sample. Proteins within the microparticle-enriched sample are then extracted before digestion with a proteolytic enzyme such as trypsin to obtain a digested sample comprising a plurality of peptides. The digested sample is then subjected to a peptide purification/concentration step before analysis to obtain a proteomic profile of the sample, e.g., by liquid chromatography and mass spectrometry. In some embodiments, the purification/concentration step comprises reverse phase chromatography (e.g., ZIPTIP pipette tip with 0.2 μL C18 resin, from Millipore Corporation, Billerica, MA).

### 3. Clinical Data

Diagnostic methods also include clinical data. Clinical data can be of different types, and collected at different stages of pregnancy or pre-pregnancy.

### a) Pre-Pregnancy Maternal Data

Pre-pregnancy data refers to data about the subject that can be obtained (but does not have to be obtained) before pregnancy. It includes, without limitation, (1) social determinants of health, (2) prior episode of preterm birth, (3) prior episode of preeclampsia, (4) prior stillbirth, (5) prior miscarriage, (6) presence or absence of a chronic health condition, (7) a prior gynecological complication, (8) race/ethnicity, (9) smoking history, (10) drug use history, and (11) body mass index. Social determinants of health can be selected from, among others: access to healthcare; healthcare insurance status; social status; social support networks; educational attainment; employment/working conditions; social environments; physical environments; community exposure to pollutants; personal health practices and coping skills; healthy child development; and culture. In some embodiments, pre-pregnancy maternal data can refer to data commonly found in clinical medical records.

### b) Conception Data

Conception data refers to data concerning the circumstances of conception. Conception data includes, without limitation, natural fertilization status, in vitro fertilization status, artificial conception status, surrogate mother status, and time interval from prior pregnancy.

### c) Pregnancy Maternal Data

Pregnancy maternal data refers to any clinical data about the subject or her pregnancy. Typically, such data is collected by a physician or other health worker. Such clinical data includes, without limitation, physician clinical observations, results of physical examinations, standard blood and/or urine testing values, ultrasound assessments, presence or absence of bleeding, blood pressure data, presence or absence of gestational diabetes, and symptoms of preterm labor.

### d) Radiographic Data

Radiographic data may be considered a kind of pregnancy maternal data. It refers to results of any radiographic imaging of a subject, such as X-ray, MRI, ultrasound, CT scan, etc. Such results may indicate characteristics of the fetus, including anatomical deformities or lack thereof, position in the uterus, etc., and also may indicate anatomical characteristics of the mother including cervical length.

### e) NIPT Fetal Genomic Data

NIPT stands for “non-invasive prenatal testing”. NIPT testing involves analysis of fetal DNA for genetic markers and evidence of genetic abnormalities. Such abnormalities include aneuploidy and polyploidy, and also can include presence or absence of specific genetic disorders. Another factor determinable by NIPT is fetal sex.

### 4. Comparand Output Dataset

Another dataset useful in developing predictive models are comparand output datasets. Comparand output datasets comprise, as an output, data that compares measures of features taken at two different time points in pregnancy. For example, one comparand output dataset compares first trimester microparticle data with second trimester microparticle data. The measure of difference can involve any function that compares the two datasets. Such functions include linear, logarithmic or normalized differences. One such function is an arithmetic difference in the measures of the features between the two datasets.

Other embodiments include the following: Measuring the relative change from presence to absence of a multitude of biomarkers (or single biomarkers); measuring linear, logarithmic or normalized changes of biomarker quantification values of multitude of biomarkers (or single biomarkers); measuring the increase, decrease, or lack of change in a ratio of single or multiple biomarker quantification values; measuring the relative rate of change of a multitude of biomarkers (or single biomarkers); measuring the change in the result of a formula which could be derived from the addition, subtraction, multiplication, division, logistic regression, and/or logarithmic change of values obtained via such methods. Normalization can occur as a function of the number of circulated microparticles detected, the protein yield of the microparticles, the number of placental-derived circulating microparticles detected, and/or the protein yield of the placental-derived microparticles.

C. Molecular Biomarkers and their Detection

### 1. Biomarkers

The term “biomarker” is used herein to refer to any feature associated with a gestational outcome. It includes both clinical biomarkers and molecular biomarkers. A molecular biomarker is a biological molecule, the presence, form or amount of which exhibits a statistically significant difference between two states. Accordingly, biomarkers are useful, alone or in combination, for classifying a subject into one of a plurality of groups. Biomarkers may be naturally occurring or non-naturally occurring. For example, a biomarker may be a naturally occurring protein or a non-naturally occurring fragment of a protein. Fragments of a protein can function as a proxy or surrogate peptide for the protein or as stand-alone biomarkers.

A variety of molecular biomarkers for adverse gestational outcomes are known. These include, without limitation, those biomarkers described in WO 2014/105985 (Jul. 3, 2014; Ezrin et al.), WO 2017/096405 (Jun. 8, 2017, Brohman et al.), WO 2019/152741 (Aug. 8, 2019, Rosenblatt et al.), and WO 2019/152745 (Aug. 8, 2019, Brohman et al.), incorporated herein by reference in its entirety.

The biomarkers can be detected using any suitable protein detection system such as de novo sequencing of proteins from microparticles isolated from a sample (e.g., blood) taken from a pregnant woman. Proteins can be sequenced by mass spectrometry, e.g., single or double (MS/MS) mass spectrometry. Both parent proteins and peptide fragments of parent proteins are useful as biomarkers of gestational outcome. Unless otherwise specified, a named protein biomarker encompasses detection by surrogate, e.g., fragments of the protein.

Proteins, e.g., peptides, detected by mass spectrometry are analyzed to identify those that are up-regulated (increased in amounts) or down-regulated (decreased in amounts) compared with controls. Proteins showing statistically significant differential expression are further analyzed to identify the parent protein. Such proteins can be identified in a protein database such as SwissProt.

In certain embodiments, biomarkers are analyzed as a panel comprising a plurality of the biomarkers. A panel can exist as a conceptual grouping, as a composition of matter (e.g., comprising purified biomarkers, or as an article, such as solid support attached to a capture reagent such as an antibody, further bound to the biomarker. The solid support can be, for example, one or more solid particles, such as beads, or a chip in which biomarkers are attached in an array format.

In certain embodiments, biomarkers can be comprised in a composition in which the peptide biomarker is paired with and a stable isotopic standard of the peptide. Such compositions are useful for detection in multiple reaction monitoring mass spectrometry.

For purposes of mass spectrometry, proteins can be detected intact, or through fragmentation, e.g., in multiple reaction monitoring (MRM). In such cases, proteins can be fragmented proteolytically before analysis. Proteolytic fragmentation includes both chemical and enzymatic fragmentation. Chemical fragmentation includes, for example, treatment with cyanogen bromide. Enzymatic fragmentation includes, for example, digestion with proteases such as trypsin, chymotrypsin, LysC, ArgC, GluC, LysN and AspN. Detection of these protein fragments, or fragmented forms of them produced in mass spectrometry, can function as surrogates for the full protein.

Biomarkers can be detected and quantified by any method known in the art. This includes, without limitation, immunoassay, chromatography, mass spectrometry, electrophoresis and surface plasmon resonance.

Detection of a biomarker includes detection of an intact protein, or detection of surrogate for the protein, such as a fragment.

Immunoassay methods include, for example, radioimmunoassay, enzyme-linked immunosorbent assay (ELISA), sandwich assays and Western blot, immunoprecipitation, immunohistochemistry, immunofluorescence, antibody microarray, dot blotting, and FACS.

Chromatographic methods include, for example, affinity chromatography, ion exchange chromatography, size exclusion chromatography/gel filtration chromatography, hydrophobic interaction chromatography and reverse phase chromatography, including, e.g., HPLC.

### 2. Mass Spectrometry

In some embodiments, detecting the level (e.g., including detecting the presence) of a microparticle-associated protein is accomplished using a liquid chromatography/mass spectrometry (LCMS)-based proteomic analysis. In an exemplary embodiment the method involves subjecting a sample to size exclusion chromatography and collecting the high molecular weight fraction (e.g., by size-exclusion chromatography) to obtain a microparticle-enriched sample. The microparticle-enriched sample is then disrupted (using, for example, chaotropic agents, denaturing agents, reducing agents and/or alkylating agents) and the released contents subjected to proteolysis. The disrupted microsome preparation, containing a plurality of peptides, is then processed using the tandem column system (e.g., as disclosed in WO 2020/097593 (Rosenblatt et. al.) and incorporated herein by reference in its entirety) prior to peptide analysis by mass spectrometry, to provide a proteomic profile of the sample. The methods disclosed herein such as in WO 2020/097593 avoid the necessity of protein concentration/purification, buffer exchange and liquid chromatography steps associated with previous methods.

Proteins in a sample can be detected by mass spectrometry. Mass spectrometers typically include an ion source to ionize analytes, and one or more mass analyzers to determine mass. Mass analyzers can be used together in tandem mass spectrometers. Ionization methods include, among others, electrospray or laser desorption methods. Mass analyzers include quadrupoles, ion traps, time-of-flight instruments and magnetic or electric sector instruments. In certain embodiments, the mass spectrometer is a tandem mass spectrometer (e.g., “MS-MS”) that uses a first mass analyzer to select ions of a certain mass and a second mass analyzer to analyze the selected ions. One example of a tandem mass spectrometer is a triple quadrupole instrument, the first and third quadrupoles act as mass filters, and an intermediate quadrupole functions as a collision cell. Mass spectrometry also can be coupled with up-stream separation techniques, such as liquid chromatography or gas chromatography. So, for example, liquid chromatography coupled with tandem mass spectrometry can be referred to as “LC-MS-MS”.

Mass spectrometers useful for the analyses described herein include, without limitation, Altis™ quadrupole, Quantis™ quadrupole, Quantiva™ or Fortis™ triple quadrupole from ThermoFisher Scientific, the 8050 or 8060 triple quadruploes from Shimadzu, the Xevo TQ-XS™ triple quadrupole from Waters, QSight™ Triple Quad LC/MS/MS from Perkin Elmer, and others.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods and compositions disclosed herein. Suitable peptide MS and MS/MS techniques and systems are known in the art (see, e.g., Methods in Molecular Biology, vol. 146: “Mass Spectrometry of Proteins and Peptides”, by Chapman, ed., Humana Press 2000; Kassel & Biemann (1990) Anal. Chem. 62:1691-1695; Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more peptides. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS).

Selected reaction monitoring is a mass spectrometry method in which a first mass analyzer selects a protein of interest (precursor), a collision cell fragments the protein into product fragments and one or more of the fragments is detected in a second mass analyzer. The precursor and product ion pair is called an SRM “transition”. The method is typically performed in a triple quadrupole instrument. When multiple fragments of a protein are analyzed, the method is referred to as Multiple Reaction Monitoring Mass Spectrometry (“MRM-MS”).

Typically, protein samples are digested with a proteolytic enzyme, such as trypsin, to produce peptide fragments. Heavy isotope labeled analogues of certain of these peptides are synthesized as standards. These standards are referred to as Stable Isotopic Standards or “SIS”. SIS peptides are mixed with a protease-treated sample. The mixture is subjected to triple quadrupole mass spectrometry. Peptides corresponding to the daughter ions of the SIS standards and the target peptides are detected with high accuracy, in either the time domain or the mass domain. Usually, a plurality of the daughter ions is used to unambiguously identify the presence of a parent ion, and one of the daughter ions, usually the most abundant, is used for quantification. SIS peptides can be synthesized to order, or can be available as commercial kits from vendors such as, for example, e.g., ThermoFisher (Waltham, MA) or Biognosys (Zurich, Switzerland).

As used herein, the terms “multiple reaction monitoring (MRM)” or “selected reaction monitoring (SRM)” refer to a MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, the assay can include standards that correspond to the analytes of interest (e.g., peptides having the same amino acid sequence as that of analyte peptides), but differ by the inclusion of stable isotopes. Stable isotopic standards (SIS) can be incorporated into the assay at precise levels and used to quantify the corresponding unknown analyte. Additional levels of specificity are contributed by the co-elution of the unknown analyte and its corresponding SIS, and by the properties of their transitions (e.g., the similarity in the ratio of the level of two transitions of the analyte and the ratio of the two transitions of its corresponding SIS).

Accordingly, detection of a protein target by MRM-MS involves detection of one or more peptide fragments of the protein, typically through detection of a stable isotope standard peptide against which the peptide fragment is compared. Typically, an SIS will, itself, be fragmented in a collision cell as the original digested fragment, and one or more of these fragments is detected by the mass spectrometer.

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS); APPI-MS/MS; and APPI-(MS)n. Peptide ion fragmentation in tandem MS (MS/MS) arrangements can be achieved using techniques known in the art, such as, e.g., collision induced dissociation (CID). As described herein, detection and quantification of biomarkers by mass spectrometry can involve multiple reaction monitoring (MRM), such as described, inter alia, by Kuhn et al. (2004) Proteomics 4:1175-1186. Scheduled multiple-reaction-monitoring (Scheduled MRM) mode acquisition during LC-MS/MS analysis enhances the sensitivity and accuracy of peptide quantitation. Anderson and Hunter (2006) Mol. Cell. Proteomics 5(4):573-588. Mass spectrometry-based assays can be advantageously combined with upstream peptide or protein separation or fractionation methods, such as, for example, with the tandem column system described herein.

### IV. Data Analysis

As used herein, the term “analysis” refers to any algorithm that transforms inputs into outputs. Analyses include, without limitation, statistical analyses, machine learning analyses and neural net analyses. The term “data” may include data received from various data sources, metadata associated with the data, and/or a combination of both data and metadata.

### A. Measurements

A measurement of a variable, such as sequencing reads mapping to a position, can be any combination of numbers and words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category, e.g., category into which the sequencing read is classified. Measurements of a variable on an ordinal scale produce a ranking, such as “first”, “second”, “third”. Measurements on a ratio scale include, for example, any measure on a predefined scale, absolute number of reads, normalized or estimated numbers, as well as statistical measurements such as frequency, mean, median, standard deviation, or quantile. Measurements that involve quantification are typically determined at the ratio scale level.

### B. Analysis

In some embodiments, analysis may involve statistical analysis of a sufficiently large number of samples to provide statistically meaningful results. Some example methods, or tools, include, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression, stepwise regression, ridge regression, lasso regression, elasticnet regression) or non-parametric analysis (e.g., Wilcoxon rank-sum test, Wilcoxon sign-rank test, sign test). Such methods produce models or classifiers which one can use to classify a particular biomarker profile into a particular state.

Statistical analysis can be operator implemented or implemented by machine learning.

In some variations, analysis may involve implementing machine learning techniques including linear and non-linear models, e.g., processes such as CART—classification and regression trees), artificial neural networks such as back propagation networks, discriminant analyses (e.g., Bayesian classifier or Fischer analysis), logistic classifiers, and support vector classifiers (e.g., support vector machines).

Classification models, also referred to as models, can be generated by mathematical analysis, including by machine learning techniques that perform analysis of datasets of biomarker measurements derived from subjects classed into one or another group.

Diagnostic tests are characterized by sensitivity (percentage classified as positive that are true positives) and specificity (percentage classified as negative that are true negatives). The relative sensitivity and specificity of a diagnostic test can involve a trade-off—higher sensitivity can mean lower specificity, while higher specificity can mean lower sensitivity. These relative values can be displayed on a receiver operating characteristic (ROC) curve. The diagnostic power of a set of variables, such as biomarkers, is reflected by the area under the curve (AUC) of an ROC curve.

In some embodiments, the classifiers of this disclosure have a sensitivity, specificity, positive predictive value or negative predictive value of at least 85%, at least 90%, at least 95%, at least 98%, or at least 99%. Classifiers of this disclosure have an AUC of at least 0.6, at least 0.7, at least 0.8, at least 0.9 or at least 0.95.

Classification can be based on a measurement of a biomarker being above or below a selected cutoff level. In certain embodiments, a cutoff value is obtained by measuring biomarker levels in a plurality of positive and negative reference samples, e.g., at least 10, 20, 50, 100 or 200 samples of each type. A cutoff can be established with respect to a measure of central tendency, such as mean, median or mode in the negative samples. A measure of deviation from this measure of central tendency can be used to set the cutoff. For example, the cutoff can be set based on variance or standard deviation. For example, the cutoff can be based on Z score, that is, a number of standard deviations above a mean of normal samples, for example one standard deviation, two standard deviations, three standard deviations or four standard deviations. For example, cutoff values can be selected so that the diagnostic test has at least 80%, 90%, 95%, 98%, 99%, 99.5%, or 99.9% sensitivity, specificity and/or positive predictive value.

Numerically, an increased risk is associated with an odds ratio of over 1.0, preferably over 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 for gestational outcome.

### C. Model Generation

The execution of a model produces an inference, also referred to as a prediction, classification or diagnosis.

Models can be created by analytic methods. Analytic methods can include any useful methodology including, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test).

Machine learning involves training machine learning models on training data sets comprising data from a plurality of test subjects. Machine learning models are trained on the training dataset to generate models that predict the gestational outcome of an individual based on sequence data or information derived therefrom. Predicted gestational outcome can be translated into recommendations to the subject about therapeutic interventions to be taken.

The machine learning model can be any suitable supervised machine learning model, parametric or non-parametric. Machine learning models include, without limitation, artificial neural networks (e.g., back propagation networks), decision trees (e.g., recursive partitioning processes, CART), random forests, discriminant analyses (e.g., Bayesian classifier or Fischer analysis), linear classifiers (e.g., multiple linear regression (MLR), partial least squares (PLS) regression, principal components regression (PCR)), mixed or random-effects models, non-parametric classifiers (e.g., k-nearest neighbors), support vector machines, and ensemble methods (e.g., bagging, boosting).

Methods for generating models to predict gestational outcome can comprise the following operations. A dataset as described above is provided. The dataset includes, for each of a plurality of subjects, raw or processed data. The data set is used as a training dataset to train a machine learning model to produce one or more models that predict gestational outcome of a subject based on biomarkers identified from the data.

Biomarkers can be individual features used by the model in making an inference (i.e. prediction or diagnosis) of the category in question. For example, of thousands of features used in the original training dataset, the model may use no more than any of 1, 5, 10, 50, 100 or 500 features in determining the classification.

### D. Longitudinal Model

Longitudinal models make predictions at a plurality of different time points in pregnancy, for example, post conception (e.g., within a month of conception), during the first trimester (but typically after the first month), and during the second trimester. Each prediction uses a model generated using data collectable up to that time point. So, for example, a post-conception prediction model may use data on pre-pregnancy maternal data and conception data. A first trimester model may add to this clinical data and microparticle data received later in the first trimester (e.g., 10-12 weeks). A second trimester model may add to this clinical data and microparticle data received later in the second trimester (e.g., 22-24 weeks). Accordingly, a plurality of models can be developed to make predictions of gestational outcomes for a subject over the course of pregnancy, e.g., post-conception stage, first trimester and second trimester.

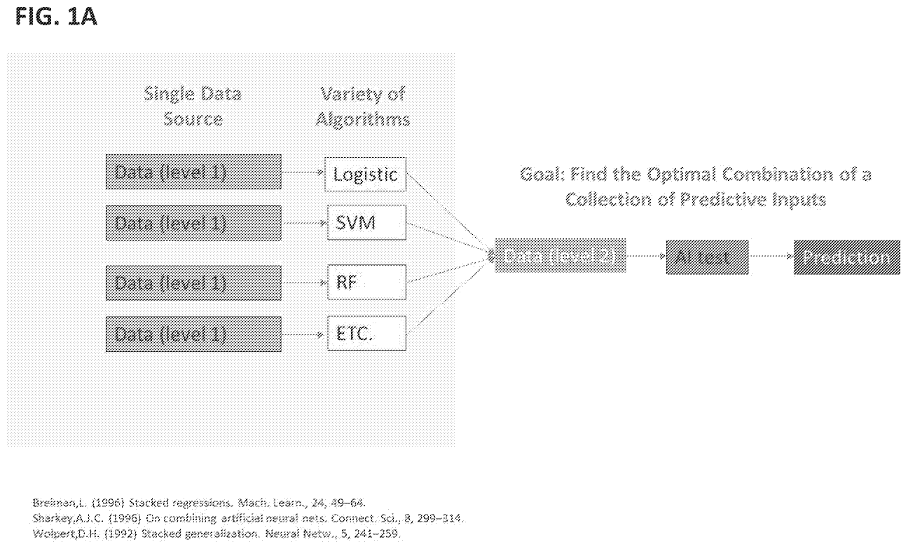

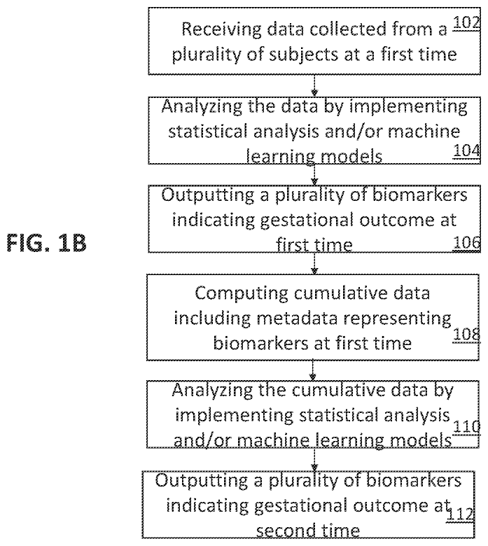

Referring to FIG. 1A, building a longitudinal model for gestational outcome involves a two-step process. In a first step, a plurality of datasets (“Data (level 1)”), each containing a different class of data, are independently analyzed to identify predictive biomarkers from each set. In a second step, the predictive biomarkers identified from each analysis are combined into one (or more) meta-dataset that are, themselves, subjected to analysis, to develop a predictive model. This can be done to develop models that are applicable at the various stages of pregnancy desired, e.g., post-conception, first trimester, second trimester.

### 1. Model Building

### a) First-Level Analysis

FIG. 1B is a flowchart illustrating aspects of model building according to some embodiments. At 102, the method can include receiving data collected from a plurality of subjects at a first time. Each subject may be classified according to a gestational outcome, which can be the ultimate object of prediction or inference. A suitable number of subjects may be chosen to produce statistically reliable results.

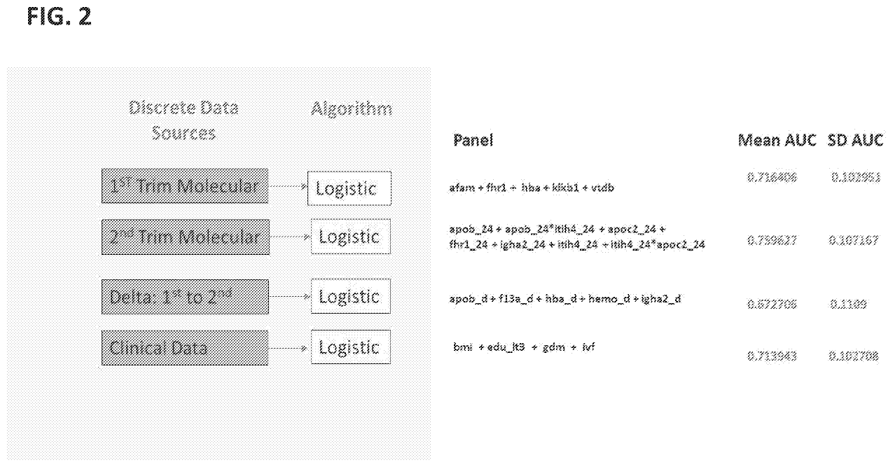

Data from different sources are received from each subject and assembled into datasets. Referring to FIG. 2, exemplary datasets can include first trimester microparticle data, second trimester microparticle data, comparand output data (that measures differences between the first and second trimester molecular data) and clinical data. Clinical data can, itself, be divided into separate datasets based on the type of clinical data (e.g., maternal status data, conception data, physical exam data, radiological data) and the timing of receipt (e.g., clinical data received post conception, during the first trimester or during the second trimester). Referring back to FIG. 1B, at 104, each of these datasets can be independently analyzed. For example, as discussed above one or more statistical analysis or machine learning models may be implemented on these datasets. At 106, the independent analyses can identify and output biomarkers that are diagnostic of a particular gestational outcome. The type of analysis for each dataset can be different, or the same. For example, one dataset can be subject to logical regression. Another, support vector machine analysis. Another, random forest. The product of these analyses is a set of biomarkers from different data sources, which have been determined to have predictive value. In this manner, a subject may be classified at a first time according to a gestational outcome.

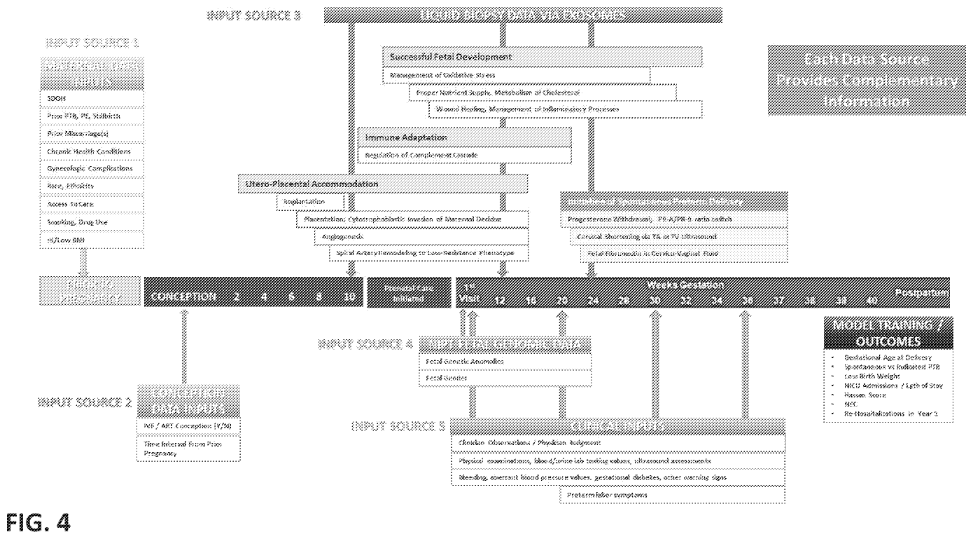

FIG. 4. shows exemplary sources of data for datasets (“Input Source”), including exemplary features of the data sources. In this figure, input sources include Maternal Data Inputs (also referred to herein as “pre-pregnancy maternal data”, a form of clinical data), conception data inputs (also a form of clinical data), liquid biopsy data via exosomes (an example of microparticle molecular data), NIPT fetal genomic data (also a form of clinical data) and clinical inputs (also a form of clinical data). Liquid biopsy data can produce biomarkers indicative of various biological functions, also indicated in FIG. 4. FIG. 4 also depicts example timing of receipt of each of the data sources. For instance, as depicted in FIG. 4, the maternal data inputs can be received pre-pregnancy, conception data inputs can be received during conception, liquid biopsy data can be received during conception during conception and during gestation, NIPT fetal genomic data and other clinical inputs can be received during gestation.

### b) Second-Level Analysis

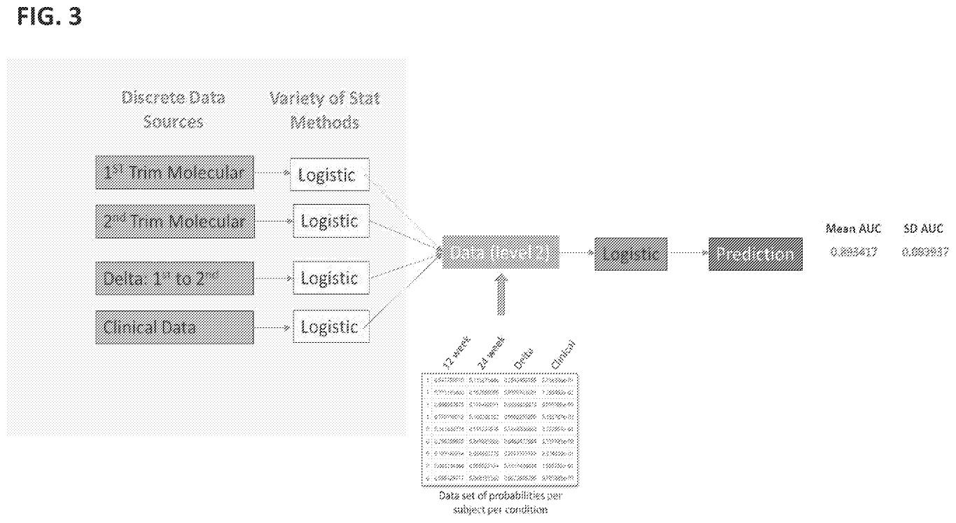

Referring to FIG. 3, in second level analysis, biomarkers identified from analyses in the first-level analysis are combined into a meta-dataset, and subject to analysis to produce a model for predicting gestational outcome. For example, the meta-dataset (“Data (level 2)”) can be subject to logistic regression to produce a predictive model. Referring back to FIG. 1B, at 108, the cumulative data that combines the biomarkers identified from the analyses in the first-level analysis with the first trimester data, second trimester data, the comparand output data, and the clinical data can be computed. At 110, the cumulative data can be analyzed to output a plurality of biomarkers that indicate a gestational outcome at a second time.

It should be readily understood that a plurality of models can be generated for making predictions at each of a plurality of different times during pregnancy, e.g., post-conception, first trimester, and/or second trimester by implementing the first-level analysis and the second-level analysis. Each model will use biomarkers available up to that point of the pregnancy. For example, a post-conception predictive model (e.g., a predictive model generated post-conception) can include biomarkers identified from analysis of maternal status data and conception status data. A first trimester model can include biomarkers identified from maternal status, conception status first-trimester clinical data and first-trimester microparticle data, but not biomarkers identified from second trimester microparticle data. A second trimester model can include biomarkers identified from all of these, as well as biomarkers identified from second trimester clinical data, and comparand output data (e.g., data comparing first trimester microparticle data with second trimester microparticle data).

It should be readily understood that although the longitudinal model building is described herein as incorporating first trimester data and second trimester data, data received and collected at any suitable point during the pregnancy may be incorporated into model building. The first-level analysis may be implemented on the data collected and received at a first time point (e.g., at t=t₁) and the second-level analysis may be implemented on the data collected and received at any subsequent time points (e.g., at t=t₁+n, where n=1, 2, 3, . . . etc.).

### 2. Model Application

Each of the models developed through second-level analysis can be applied to test subjects to predict gestational outcome. In doing so, data is received from the test subject that includes biomarkers used in the second-level model. So for example, where educational attainment is determined to be a biomarker for preterm birth, this data may be collected, while data on other maternal status elements, may not. Data is received through the time point in pregnancy where the prediction is to be made, for example, post conception, first trimester and second trimester.

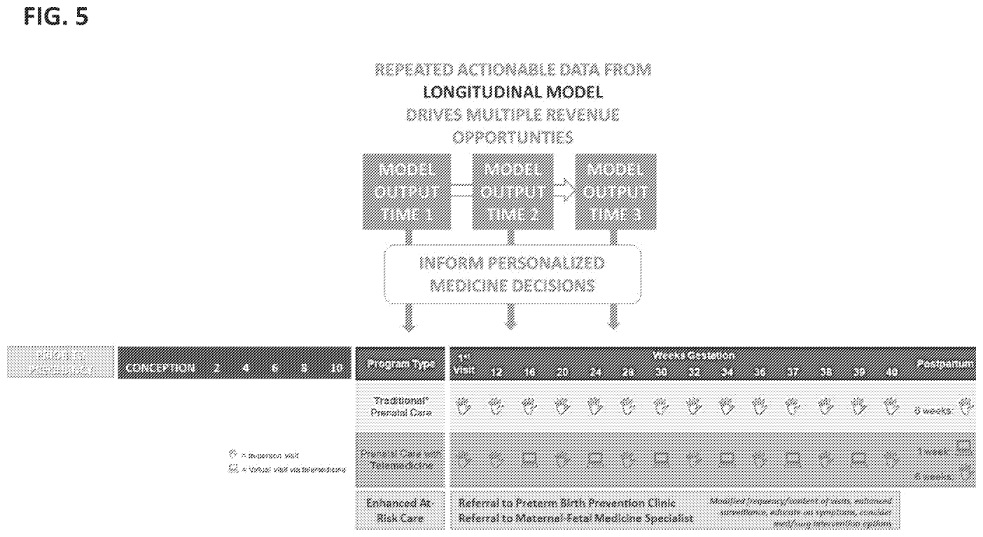

Referring to FIG. 5, a health care provider can present to a pregnant subject exemplary therapeutic tracks/treatment tracks based on whether the subject is classified into high, medium and low preterm birth risk states, at three different time points. Pre-pregnancy maternal data and conception data are collected. Using this data, a post-conception predictive model outputs predictions for one or a number of gestational outcomes (Model Output Time 1). As pregnancy progresses, more data is collected during the first trimester. Using this data, first trimester gestational outcomes are predicted (Model Output Time 2). More data is collected during the second trimester. Using this data, second trimester gestational outcomes are predicted (Model Output Time 3). The predictions may be given as risk scores for particular adverse gestational outcomes. As discussed below, each of these predictions can be followed by tracking the subject into one of a plurality of different therapeutic tracks/treatment tracks.

### E. Validation

A model may be subsequently validated using a validation dataset. Validation datasets typically include data on the same features as the training dataset. The model is executed on the training dataset and the number of true positives, true negatives, false positives and false negatives is determined, as a measure of performance of the model.

The model can then be tested on a validation dataset to determine its usefulness. Typically, a learning model will generate a plurality of models. In certain embodiments, models can be validated based on fidelity to standard clinical measures used to diagnose the condition under consideration. One or more of these can be selected based on its performance characteristics.

### V. Methods of Treatment

Predictions made using the models described herein can be used to guide treatment of subjects. For example, based on the predicted risk level for an adverse gestational outcome, Individual subjects can be placed on different treatment tracks. The tracks can include, low risk (normal), medium risk and high risk for a particular adverse gestational outcome, such as preterm birth. Each track will include different treatment regimens at each of the time points. Because subjects can be tested for risk at a plurality of time points, the treatment track can change if the predicted risk changes at a particular time point. So, a subject that is medium risk at an earlier time point, may move to a low risk time point later. And a subject a low risk at one point, may move to high risk at a later time point.

Referring to FIG. 5, three different standard of care tracks/treatment tracks can be provided. In a low risk, or normal, track, a subject can be scheduled for in-person visits every six weeks. In a medium risk track, a subject can have, in addition, weekly telemedicine visits with a healthcare provider. In a high risk track, a subject can receive enhanced care, which may include referral to a preterm birth prevention clinic and/or referral to maternal-fetal medicine specialist.

Specific medical or surgical interventions for high risk of preterm birth can include, progestogen supplementation, low-dose aspirin, cervical cerclage, administration of antenatal corticosteroids and tocolytics.

### VI. Kits and Other Articles of Manufacture

In another embodiment, provided herein are articles of manufacture, e.g., kits of reagents useful in detecting in a sample biomarkers for increased risk of gestational outcome, in particular, gestational outcome requiring delivery in <=35 weeks gestation. Reagents capable of detecting protein biomarkers include but are not limited to antibodies. Antibodies capable of detecting protein biomarkers are also typically directly or indirectly linked to a molecule such as a fluorophore or an enzyme, which can catalyze a detectable reaction to indicate the binding of the reagents to their respective targets.

In some embodiments, the kits further comprise sample processing materials comprising a high molecular weight gel filtration composition (e.g., agarose such as SEPHAROSE) in a low volume (e.g., 1 ml, 3 ml, 5 ml, 10 ml) vertical column for rapid preparation of a microparticle-enriched sample from plasma. For instance, the microparticle-enriched sample can be prepared at the point of care before freezing and shipping to an analytical laboratory for further processing.

In some embodiments, the kits further comprise instructions for assessing risk of gestational outcome, in particular, gestational outcome requiring delivery in <=35 weeks gestation. As used herein, the term “instructions” refers to directions for using the reagents contained in the kit for detecting the presence (including determining the expression level) of a protein(s) of interest in a sample from a subject. The proteins of interest may comprise one or more biomarkers of gestational outcome. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination.

In another embodiment, a kit comprises a container containing one or a plurality of stable isotope standard (SIS) peptides corresponding to peptide biomarkers, e.g., peptides produced from protease (e.g., trypsin) digestion of biomarker proteins. In another embodiment, a majority or all of the SIS peptides correspond to the biomarker peptides. In another embodiment, the kit further comprises the biomarker peptides which the SIS peptides correspond.

In another embodiment, provided is a composition of matter that includes protein biomarkers of gestational outcome and, for a plurality of those biomarkers, a corresponding stable isotope standard peptide. This can be prepared by combining a sample comprising proteins isolated from microparticles, with stable isotope standard peptides.

### VII. Systems

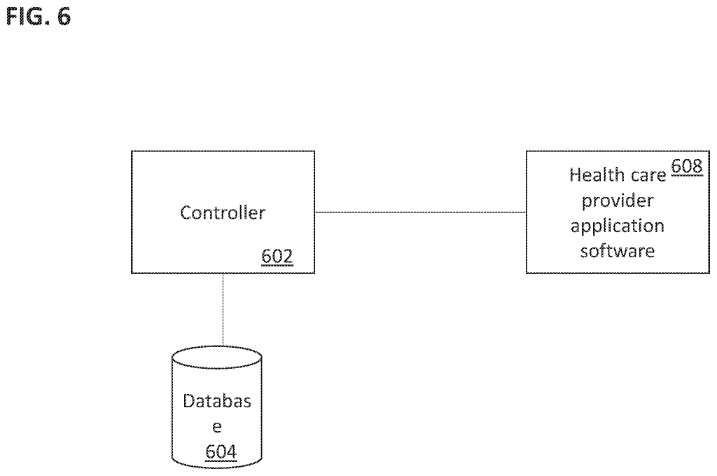

FIG. 6 shows an exemplary system for predicting adverse gestational outcome. The system may access and/or retrieve data from database 604. A controller 602 may implement statistical analysis and/or machine learning models using the data retrieved from database 604. In some variations, the controller 602 may build longitudinal model(s) as described above using the data retrieved from the database 604. The predictions from the longitudinal model(s) may be transmitted to a health provider application 608 being implemented on a suitable computing device. In some variations, the predictions from the longitudinal model(s) (e.g., biomarkers outputted from the models) may be stored in the database 604. In some variations, these predictions may be accessed from the database 604 at a future time to improve the accuracy of the longitudinal model(s).

In some variations, the controller 602 may include one or more servers and/or one or more processors running on a cloud platform (e.g., Microsoft Azure®, Amazon® web services, IBM® cloud computing, etc.). The server(s) and/or processor(s) may be any suitable processing device configured to run and/or execute a set of instructions or code, and may include one or more data processors, image processors, graphics processing units, digital signal processors, and/or central processing units. The server(s) and/or processor(s) may be, for example, a general purpose processor, a Field Programmable Gate Array (FPGA), an Application Specific Integrated Circuit (ASIC), and/or the like.

In some variations, the controller 602 may include a processor (e.g., CPU). The processor may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like. In some variations, the controller 602 may include one or more modules (e.g., modules in a software code and/or modules stored in a memory) that, when executed by the processor, can be configured to classify gestational outcome of a subject.

The output of the longitudinal model(s) and first trimester and second trimester data associated with a plurality of subjects may be stored in the database 604. The controller 602 can be communicably coupled to the database 604. The database 604 may be accessed at any suitable time to improve the longitudinal model(s) implemented by the controller 602. In some variations, the database 604 may be stored in a memory device such as a random access memory (RAM), a memory buffer, a hard drive, an erasable programmable read-only memory (EPROM), an electrically erasable read-only memory (EEPROM), a read-only memory (ROM), Flash memory, and the like. In some variations, the database 604 may be stored on a cloud-based platform such as Amazon web Services®.

The output of the longitudinal model(s) may be accessible to health care providers via an application software 608 executable on a computing device. Some non-limiting examples of the computing device include computers (e.g., desktops, personal computers, laptops etc.), tablets and e-readers (e.g., Apple iPad®, Samsung Galaxy® Tab, Microsoft Surface®, Amazon Kindle®, etc.), mobile devices and smart phones (e.g., Apple iPhone®, Samsung Galaxy®, Google Pixel®, etc.), etc. In some variations, the application software 608 (e.g., web apps, desktop apps, mobile apps, etc.) may be pre-installed on the computing device. Alternatively, the application software 608 may be rendered on the computing device in any suitable way. For example, in some variations, the application software 608 (e.g., web apps, desktop apps, mobile apps, etc.) may be downloaded on the computing device from a digital distribution platform such as an app store or application store (e.g., Chrome® web store, Apple® web store, etc.). Additionally or alternatively, the computing device may render a web browser (e.g., Google®, Mozilla®, Safari®, Internet Explorer®, etc.) on the computing device. The web browser may include browser extensions, browser plug-ins, etc. that may render the application software 608 on the computing device. In yet another alternative variation, the browser extensions, browser plug-ins, etc. may include installation instructions to install the application software 608 on the computing device.

The output of the longitudinal model(s) may be accessed by any user (e.g., patient, health care providers, other clinicians, etc.) via the application software 608 in real-time. For example, the health care providers may access the output of the longitudinal model(s) via the application software 608 in real-time. Additionally, in some variations, the application software 608 may allow health care providers to access, review, and/or edit data (e.g., first trimester data, second trimester data etc.) in real-time. The output of the longitudinal model(s) may be displayed on the display of the computing device.

Data can be transmitted electronically, e.g., over the Internet. Electronic communication can be, for example, over any communications network include, for example, a high-speed transmission network including, without limitation, Digital Subscriber Line (DSL), Cable Modem, Fiber, Wireless, Satellite and, Broadband over Powerlines (BPL). Information can be transmitted to a modem for transmission, e.g., wireless or wired transmission, to a computer such as a desktop computer. Alternatively, reports can be transmitted to a mobile device. Reports may be accessible through a subscription program in which a user accesses a website which displays the report. Reports can be transmitted to a user interface device accessible by the user. The user interface device could be, for example, a personal computer, a laptop, a smart phone or a wearable device, e.g., a watch, for example worn on the wrist.

### VIII. Communicating Results in Implementing Therapeutic Interventions

Inference models as described herein can be executed on subject data to predict (e.g., estimate risk of) a gestational outcome and/or recommendations for therapeutic track/treatment track. In one embodiment, after making an inference about a state of gestational outcome, the method can comprise developing a model for therapeutic intervention in the subject. The model can comprise, for example, a treatment track for the subject, or pharmaceutical compositions to administer to the subject to treat the condition. Such a model can be communicated to the subject, for example, transmitting the model and, optionally, the diagnosis, to a user interface of a personal computing device of the subject.

Inferences on a subject's state and/or recommendations for therapeutic intervention can be provided to subjects through an Internet website. A website can be provided which can be accessed by a subject, e.g. a customer, through a password-protected portal. The website can include a clickable icon. Upon clicking the icon, the subject can receive personalized food recommendations. Such inferences and/or recommendations can be displayed on a webpage connected to the clickable icon. Subject can receive at an Internet connected server notification that inferences and/or recommendations for the subject are available.

After therapeutic interventions are implemented, the effect of these interventions on the subject's condition can be remeasured. Such remeasurements can be used to generate updated inferences and/or recommendations as described herein.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

### Exemplary Embodiments

Embodiment A1. A method for generating a model that infers a gestational outcome in a subject in the second trimester of pregnancy comprising:
a) receiving, at a controller, a plurality of datasets comprising data on each of a plurality of subjects, wherein the plurality of datasets include at least one of:
   i) one or more datasets comprising measures of clinical data;
   ii) a dataset comprising measures of first trimester microparticle data;
   iii) a dataset comprising measures of second trimester microparticle data; or
   iv) a dataset comprising comparand output data;
   and wherein each dataset of the plurality of datasets include a gestational outcome identifier for each subject;
b) performing, via the controller, an analysis on each of the plurality of datasets, wherein the analyses identify, from each dataset, one or a plurality of dataset features that infer a gestational outcome in subject;
c) computing, via the controller, a meta-dataset that includes, for each subject, measures of a plurality of the identified features from each of the datasets and the gestational outcome identifier; and
d) generating, via the controller and based on an analysis of the meta-dataset, a model to infer a gestational outcome for a subject.

Embodiment A2. The method of embodiment A1, wherein the gestational outcome is one or more of: initiation of preterm labor, spontaneous preterm birth, birth weight, neonatal intensive care unit admission/length of stay, Hassan score, necrotizing enterocolitis (NEC) and rehospitalization within one year.

Embodiment A3. The method as in one of embodiments A1-A2, wherein the model infers an adverse gestational outcome.

Embodiment A4. The method as in one of embodiments A1-A3, wherein the gestational outcome inferred is a risk score of the gestational outcome.

Embodiment A5. The method as in one of embodiments A1-A4, wherein plurality of subjects is at least 25, at least 50, at least 200, at least 500, at least 1000 or at least 10,000.

Embodiment A6. The method as in one of embodiments A1-A5, wherein the clinical data comprises data from one or more groups consisting of (i) pre-pregnancy maternal data, (ii) conception data, (iii) pregnancy maternal data, (iv) NIPT fetal genomic data, and (v) radiographic data.

Embodiment A7. The method of embodiment A6, wherein the clinical data is comprised in a plurality of separate datasets.

Embodiment A8. The method as in one of embodiments A6-A7, wherein the datasets comprising clinical data comprise a plurality of datasets, each dataset comprising data received from a plurality of different timepoints in pregnancy.

Embodiment A9. The method as in one of embodiments A6-A8, wherein the pre-pregnancy maternal data comprises one or more of: (1) a social determinants of health, (2) prior episode of preterm birth, (3) prior episode of preeclampsia, (4) prior stillbirth, (5) prior miscarriage, (6) presence or absence of a chronic health condition, (7) a prior gynecological complication, (8) race/ethnicity, (9) smoking, (10) drug use, and (11) body mass index.

Embodiment A10. The method as in one of embodiments A6-A9, wherein the pre-pregnancy maternal data comprises one or more social determinants of health selected from access to healthcare; healthcare insurance status; social status; social support networks; educational attainment; employment/working conditions; social environments; physical environments; community exposure to pollutants; personal health practices and coping skills; healthy child development; and culture.

Embodiment A11. The method as in one of embodiments A6-A10, wherein the conception data comprises one or more of in vitro fertilization status, artificial conception status, and time interval from prior pregnancy.

Embodiment A12. The method as in one of embodiments A6-A11, wherein the pregnancy maternal data comprises one or more of: physician clinical observations, results of physical examinations, blood and/or urine testing values, ultrasound assessments, presence or absence of bleeding, blood pressure data, presence or absence of gestational diabetes, and symptoms of preterm labor.

Embodiment A13. The method as in one of embodiments A6-A12, wherein the NIPT fetal genomic data comprise one or more of fetal sex and presence or absence of fetal genetic abnormality.

Embodiment A14. The method as in one of embodiments A6-A13, wherein the first trimester microparticle data or the second trimester microparticle data comprises liquid biopsy data.

Embodiment A15. The method of embodiment A14, wherein the liquid biopsy data comprises exosome-derived data.

Embodiment A16. The method as in one of embodiments A6-A15, wherein the first trimester microparticle data or the second trimester microparticle data comprises one or more biomarkers of management of oxidative stress, proper nutrient supply, metabolism of cholesterol, wound healing, and management of inflammatory processes.

Embodiment A17. The method as in one of embodiments A6-A16, wherein the first trimester microparticle data or the second trimester microparticle data comprises biomarkers involved in the regulation of the complement cascade.

Embodiment A18. The method as in one of embodiments A6-A17, wherein the first trimester microparticle data or the second trimester microparticle data comprises one or more biomarkers related to embryo implantation, placentation, cytotrophoblastic invasion of the maternal decidua, abnormal placental development, angiogenesis and spiral artery remodeling to a low resistance phenotype.

Embodiment A19. The method as in one of embodiments A1-A18, wherein the identified features of the first and second trimester microparticle datasets are not identical.

Embodiment A20. The method as in one of embodiments A1-A19, wherein the gestational outcome identifier comprises one or more of the following indicators of preterm labor initiation: progesterone withdrawal, PR-A/PR-B ratio switch, cervical shortening via trans-abdominal or trans-vaginal ultrasound, and fetal fibronectin in cervical-vaginal fluid.

Embodiment A21. The method as in one of embodiments A1-A20, wherein the comparand output dataset compares the data by linear, logarithmic or normalized differences.

Embodiment A22. The method as in one of embodiments A1-A21, wherein the method further comprises providing, to the controller, the dataset of measures of microparticle data comprising (1) preparing a microparticle-enriched fraction from a blood sample from the pregnant subject; and (II) determining a quantitative measure of microparticle-associated proteins in the fraction.

Embodiment A23. The method of embodiment A22, wherein the first trimester data is collected between 10 and 12 weeks of pregnancy.

Embodiment A24. The method of embodiment A22, wherein the second trimester data is collected between 24 and 26 weeks of pregnancy.

Embodiment A25. The method of embodiment A22, wherein the liquid biopsy data comprise protein data.

Embodiment A26. The method of embodiment A22, wherein the blood sample is a serum sample or a plasma sample.

Embodiment A27. The method of embodiment A22, wherein the microparticle-enriched fraction is prepared using size-exclusion chromatography.

Embodiment A28. The method of embodiment A27, wherein the size-exclusion chromatography comprises elution with distilled, deionized H₂O.

Embodiment A29. The method as in one of embodiments A27-A28, wherein the size-exclusion chromatography is performed with an agarose solid phase and an aqueous liquid phase.

Embodiment A30. The method as in one of embodiments A27-A29, wherein preparing the microparticle-enriched fraction further comprises using ultrafiltration or reverse-phase chromatography.

Embodiment A31. The method as in one of embodiments A27-A30, wherein preparing microparticle-enriched fraction further comprises denaturation using urea, reduction using dithiothreitol, alkylation using iodoacetamine, and digestion using trypsin.

Embodiment A32. The method as in one of embodiments A1-A31, wherein the I analyses comprise an analysis independently selected from: regression analysis (e.g., simple regression, multiple regression, linear regression, non-linear regression, logistic regression, polynomial regression. stepwise regression, ridge regression, lasso regression, elasticnet regression) correlational, Pearson correlation, Spearman correlation, chi-square, comparison of means (e.g., paired T-test, independent T-test, ANOVA), and non-parametric analysis (e.g., Wilcoxon rank-sum test, Wilcoxon sign-rank test, sign test), as CART—classification and regression trees), artificial neural networks such as back propagation networks, discriminant analyses (e.g., Bayesian classifier or Fischer analysis), logistic classifiers, and support vector classifiers (e.g., support vector machines).

Embodiment B1. A method for inferring a gestational outcome in a subject during second trimester comprising:
a) receiving data from a pregnant subject, wherein the data comprises measures of one or a plurality of features identified from each of:
   i) one or more datasets comprising measures of clinical data;
   ii) a dataset comprising measures of first trimester microparticle data;
   iii) a dataset comprising measures of second trimester microparticle data; and
   iv) a comparand output dataset that includes first trimester data compared to second trimester data;
wherein the first trimester microparticle data and the second trimester microparticle data is received by:
   I) providing a microparticle-enriched blood sample from a pregnant female subject; and
   II) determining a measure of each of a plurality of proteins from the sample;
b) producing a meta-dataset that comprises measures of the plurality of features; and
c) executing by computer a model that infers, from measures of the one or more features in the meta-dataset, a risk of an adverse gestational outcome in the subject.

Embodiment B2. The method of embodiment B1, wherein the model is a model created by a method of embodiment A1.

Embodiment C1. A method of treating a pregnant subject comprising:
(I) during pregnancy:
   (A) inferring a gestational outcome in a pregnant subject by executing a model on a first meta-dataset that includes for the subject, measures of a plurality of diagnostic features from:
      i) one or more first datasets comprising measures of clinical data, wherein the clinical data comprises maternal data inputs and/or conception data inputs; and
   (B) based on an inference of low, average or high risk of an adverse gestational outcome, tracking the subject into one of three treatment tracks selected from: traditional prenatal care, prenatal care with telemedicine, and enhanced at risk care;
(II) during the first trimester of pregnancy, and after (I):
   (A) inferring a gestational outcome in the subject by executing a model on a second meta-dataset that includes for the subject, measures of a plurality of diagnostic features from:
      i) one or more first datasets comprising measures of clinical data, wherein the clinical data comprises maternal data inputs and/or conception data inputs; and
      ii) a dataset comprising measures of first trimester microparticle data;
      iii) optionally, a first trimester clinical dataset comprising measures of clinical data collected during the first trimester; and
   (B) based on an inference of low, average, or high risk of an adverse gestational outcome, tracking the subject into one of three treatment tracks selected from: traditional prenatal care, prenatal care with telemedicine and enhanced at risk care; and
(III) during the second trimester of pregnancy:
   (A) inferring a gestational outcome in the subject by executing a model on a third meta-dataset that includes for the subject, measures of a plurality of diagnostic features from:
      i) one or more first datasets comprising measures of clinical data, wherein the clinical data comprises maternal data inputs and/or conception data inputs; and
      ii) a dataset comprising measures of first trimester microparticle data;
      iii) optionally, a first trimester clinical dataset comprising measures of clinical data collected during the first trimester; and
      iv) a dataset comprising measures of second trimester microparticle data;
      v) a comparand output dataset comparing data from first trimester microparticle data to second trimester microparticle data;
      vi) optionally, one or more third clinical datasets comprising measures of clinical data collected during the second trimester; and
   (B) based on an inference of low, average, or high risk of an adverse gestational outcome, tracking the subject into one of three treatment tracks selected from: traditional prenatal care, prenatal care with telemedicine, and enhanced at-risk care.

Embodiment C2. The method of embodiment C1, wherein tracking the subject into one of the three treatment tracks comprise tracking via a model.

Embodiment C3. The method as in one of embodiments C1-C2, wherein enhanced at-risk care comprise one or more of:
1. Referral to Preterm Birth Prevention Clinic;
2. Referral to Maternal Fetal Medicine specialist;
3. Education on signs/symptoms of preterm labor;
4. Evaluation of medical (i.e. progestogen supplementation, low-dose aspirin) or surgical (i.e. cervical cerclage) options;
5. Modification of behaviors, lifestyle and diet to support a healthy birth outcome;
6. Increased office visits and modified content of office visits;
7. Increased surveillance via ultrasound and cervical length measurements; and
8. Preparation for acute-stage events (i.e. planning for NICU access, education on medicines that can be given upon initiation of preterm labor to extend gestation, mature the baby's lungs, and provide neuroprotective agents for the baby's brain development)

Embodiment D1. A method for treating a pregnant subject at high risk of an adverse gestational outcome comprising:
(A) receiving a meta-dataset that includes for the subject, measures of a plurality of diagnostic features from:
   i) one or more first datasets comprising measures of clinical data, wherein the clinical data comprises maternal data inputs and/or conception data inputs; and
   ii) a dataset comprising measures of first trimester microparticle data;
   iii) optionally, a first trimester clinical dataset comprising measures of clinical data collected during the first trimester; and
   iv) a dataset comprising measures of second trimester microparticle data;
   v) an output dataset comparing data from first trimester microparticle data to second trimester microparticle data;
   vi) optionally, one or more third clinical datasets comprising measures of clinical data collected during the second trimester; and
(B) executing, by computer, a model that, based on measures of the diagnostic features, predicts an adverse gestational outcome in the subject; and
(C) based on an inference of low, average, or high risk of an adverse gestational outcome, tracking the subject into one of three treatment tracks selected from: traditional prenatal care, prenatal care with telemedicine and enhanced at risk care.

Embodiment E1. A system for inferring risk of an adverse gestational outcome comprising:
a controller; and
a database communicatively coupled to the controller, the database comprising:
   (1) a meta-dataset comprising measures of features in feature data, wherein the feature data comprises:
      i) one or more first datasets comprising measures of clinical data, wherein the clinical data comprises maternal data inputs and/or conception data inputs; and
      ii) a dataset comprising measures of first trimester microparticle data;
      iii) optionally, a first trimester clinical dataset comprising measures of clinical data collected during the first trimester; and
      iv) a dataset comprising measures of second trimester microparticle data;
      v) an output dataset comparing data from first trimester microparticle data to second trimester microparticle data;
      vi) optionally, one or more third clinical datasets comprising measures of clinical data collected during the second trimester;
   (2) a classification model which, based on the measures, infers a gestational outcome in the subject; and
   (3) computer executable instructions for implementing the classification model on the meta-dataset.

Embodiment F1. A non-transitory computer readable medium comprising machine executable code, which, when executed by a computer processor, infers an adverse gestational outcome in a subject by:
(a) accessing a meta-dataset measures of features in feature data, wherein the feature data comprises
   i) one or more first datasets comprising measures of clinical data, wherein the clinical data comprises maternal data inputs and/or conception data inputs; and
   ii) a dataset comprising measures of first trimester microparticle data;
   iii) optionally, a first trimester clinical dataset comprising measures of clinical data collected during the first trimester; and
   iv) a dataset comprising measures of second trimester microparticle data;
   v) an output dataset comparing data from first trimester microparticle data to second trimester microparticle data;
   vi) optionally, one or more third clinical datasets comprising measures of clinical data collected during the second trimester; and
(b) executing a computer model on the meta-dataset set to infer an adverse gestational outcome in the subject.

Embodiment G1. A method of treating adverse gestational outcome in a subject comprising:
(a) inferring the presence of adverse gestational outcome in a subject according to a method as described herein; and
(b) administering a therapeutic intervention to the subject effective to treat the adverse gestational outcome.

Embodiment H1. A method for diagnosing and treating an adverse gestational outcome in a subject, the method comprising:
(a) receiving from a subject: (i) pre-pregnancy maternal data, (ii) conception data, (iii) pregnancy maternal data;
(b) receiving from the subject a blood sample;
(c) determining, from a microparticle-enriched portion of the blood sample, data comprising measures of protein features;
(d) generating a meta-dataset for the subject based upon the data;
(e) generating an inference of the adverse gestational outcome in the subject upon processing the meta-dataset with an inference model derived from a population of subjects; and
(f) at an output device associated with the subject, providing a therapy to the subject for the adverse gestational outcome upon processing the inference with a therapy model designed to treat the adverse gestational outcome.

Embodiment I1. A method for creating a model that infers a gestational outcome in a subject in a first trimester of pregnancy comprising:
a) receiving, at a database, a plurality of datasets comprising data on each of a plurality of subjects, wherein the datasets include:
   i) one or more datasets comprising measures of clinical data; and
   ii) a dataset comprising measures of first trimester microparticle data;
   and wherein each dataset includes a gestational outcome identifier for each subject;
b) performing, via a controller, an analysis on each of the datasets, wherein the analyses identify, from each dataset, one or a plurality of dataset features that infer a gestational outcome in subject;
c) receiving, at the database, a meta-dataset that includes, for each subject, measures of a plurality of the identified features from each of the datasets and the gestational outcome identifier; and
d) performing, via the controller, an analysis on the meta-dataset, wherein the analysis produces, from the identified features, a model that infers a gestational outcome for a subject.

Embodiment J1. A method for creating a model that infers a gestational outcome in a subject in post-conception comprising:
a) receiving, at a database, a plurality of datasets comprising data on each of a plurality of subjects, wherein the datasets include:
   i) a dataset comprising measures of pre-pregnancy maternal data; and
   ii) a dataset comprising measures of conception status data;
   and wherein each dataset includes a gestational outcome identifier for each subject;
b) performing, via a controller, an analysis on each of the datasets, wherein the analyses identify, from each dataset, one or a plurality of dataset features that infer a gestational outcome in subject;
c) receiving, at the database, a meta-dataset that includes, for each subject, measures of a plurality of the identified features from each of the datasets and the gestational outcome identifier; and
d) performing, via the controller, an analysis on the meta-dataset, wherein the analysis produces, from the identified features, a model that infers a gestational outcome for a subject.

What is claimed is:

1. A method for generating a model that infers a gestational outcome in a subject in the second trimester of pregnancy comprising:
a) receiving, at a controller, a plurality of datasets comprising data on each of a plurality of subjects, wherein the plurality of datasets include at least one of:
i) one or more datasets comprising measures of clinical data;
ii) a dataset comprising measures of first trimester microparticle data;
iii) a dataset comprising measures of second trimester microparticle data; or
iv) a dataset comprising comparand output data;
and wherein each dataset of the plurality of datasets include a gestational outcome identifier for each subject;
b) performing, via the controller, an analysis on each of the plurality of datasets, wherein the analyses identify, from each dataset, one or a plurality of dataset features that infer a gestational outcome in subject;
c) computing, via the controller, a meta-dataset that includes, for each subject, measures of a plurality of the identified features from each of the datasets and the gestational outcome identifier; and
d) generating, via the controller and based on an analysis of the meta-dataset, a model to infer a gestational outcome for a subject.

2. The method of claim 1, wherein the gestational outcome is one or more of: initiation of preterm labor, spontaneous preterm birth, birth weight, neonatal intensive care unit admission/length of stay, Hassan score, necrotizing enterocolitis (NEC) and rehospitalization within one year.

3. The method of claim 1, wherein the model infers an adverse gestational outcome.

4. The method of claim 1, wherein the gestational outcome inferred is a risk score of the gestational outcome.

5. The method of claim 1, wherein plurality of subjects is at least 25, at least 50, at least 200, at least 500, at least 1000 or at least 10,000.

6. The method of claim 1, wherein the clinical data comprises data from one or more groups consisting of (i) pre-pregnancy maternal data, (ii) conception data, (iii) pregnancy maternal data, (iv) NIPT fetal genomic data, and (v) radiographic data.

7. The method of claim 6, wherein the clinical data is comprised in a plurality of separate datasets.

8. The method of claim 6, wherein the datasets comprising clinical data comprise a plurality of datasets, each dataset comprising data received from a plurality of different timepoints in pregnancy.

9. The method of claim 6, wherein the pre-pregnancy maternal data comprises one or more of: (1) a social determinants of health, (2) prior episode of preterm birth, (3) prior episode of preeclampsia, (4) prior stillbirth, (5) prior miscarriage, (6) presence or absence of a chronic health condition, (7) a prior gynecological complication, (8) race/ethnicity, (9) smoking, (10) drug use, and (11) body mass index.

10. The method of claim 6, wherein the pre-pregnancy maternal data comprises one or more social determinants of health selected from access to healthcare; healthcare insurance status; social status; social support networks; educational attainment; employment/working conditions; social environments; physical environments; community exposure to pollutants; personal health practices and coping skills; healthy child development; and culture.

11. The method of claim 6, wherein the conception data comprises one or more of in vitro fertilization status, artificial conception status, and time interval from prior pregnancy.

12. The method of claim 6, wherein the pregnancy maternal data comprises one or more of: physician clinical observations, results of physical examinations, blood and/or urine testing values, ultrasound assessments, presence or absence of bleeding, blood pressure data, presence or absence of gestational diabetes, and symptoms of preterm labor.

13. The method of claim 6, wherein the NIPT fetal genomic data comprise one or more of fetal sex and presence or absence of fetal genetic abnormality.

14. The method of claim 6, wherein the first trimester microparticle data or the second trimester microparticle data comprises liquid biopsy data.

15. The method of claim 14, wherein the liquid biopsy data comprises exosome-derived data.

16. The method of claim 6, wherein the first trimester microparticle data or the second trimester microparticle data comprises one or more biomarkers of management of oxidative stress, proper nutrient supply, metabolism of cholesterol, wound healing, and management of inflammatory processes.

17. The method of claim 6, wherein the first trimester microparticle data or the second trimester microparticle data comprises biomarkers involved in the regulation of the complement cascade.

18. The method of claim 6, wherein the first trimester microparticle data or the second trimester microparticle data comprises one or more biomarkers related to embryo implantation, placentation, cytotrophoblastic invasion of the maternal decidua, abnormal placental development, angiogenesis and spiral artery remodeling to a low resistance phenotype.

19. The method of claim 1, wherein the identified features of the first and second trimester microparticle datasets are not identical.

20. The method of claim 1, wherein the gestational outcome identifier comprises one or more of the following indicators of preterm labor initiation: progesterone withdrawal, PR-A/PR-B ratio switch, cervical shortening via trans-abdominal or trans-vaginal ultrasound, and fetal fibronectin in cervical-vaginal fluid.

21. The method of claim 1, wherein the comparand output dataset compares the data by linear, logarithmic or normalized differences.

22. The method of claim 1, wherein the method further comprises providing, to the controller, the dataset of measures of microparticle data comprising (I) preparing a microparticle-enriched fraction from a blood sample from the pregnant subject; and (II) determining a quantitative measure of microparticle-associated proteins in the fraction.

23. The method of claim 22, wherein the first trimester data is collected between 10 and 12 weeks of pregnancy.

24. The method of claim 22, wherein the second trimester data is collected between 24 and 26 weeks of pregnancy.

25. The method of claim 22, wherein the liquid biopsy data comprise protein data.

26. The method of claim 22, wherein the blood sample is a serum sample or a plasma sample.

27. The method of claim 22, wherein the microparticle-enriched fraction is prepared using size-exclusion chromatography.

28. The method of claim 27, wherein the size-exclusion chromatography comprises elution with distilled, deionized H₂O.

29. The method of claim 27, wherein the size-exclusion chromatography is performed with an agarose solid phase and an aqueous liquid phase.

30. The method of claim 27, wherein preparing the microparticle-enriched fraction further comprises using ultrafiltration or reverse-phase chromatography.

31. The method of claim 27, wherein preparing microparticle-enriched fraction further comprises denaturation using urea, reduction using dithiothreitol, alkylation using iodoacetamine, and digestion using trypsin.

32. The method of claim 1, wherein the I analyses comprise an analysis independently selected from: regression analysis (e.g., simple regression, multiple regression, linear regression, non-linear regression, logistic regression, polynomial regression. stepwise regression, ridge regression, lasso regression, elasticnet regression) correlational, Pearson correlation, Spearman correlation, chi-square, comparison of means (e.g., paired T-test, independent T-test, ANOVA), and non-parametric analysis (e.g., Wilcoxon rank-sum test, Wilcoxon sign-rank test, sign test), as CART—classification and regression trees), artificial neural networks such as back propagation networks, discriminant analyses (e.g., Bayesian classifier or Fischer analysis), logistic classifiers, and support vector classifiers (e.g., support vector machines).

33. A method for inferring a gestational outcome in a subject during second trimester comprising:
a) receiving data from a pregnant subject, wherein the data comprises measures of one or a plurality of features identified from each of:
i) one or more datasets comprising measures of clinical data;
ii) a dataset comprising measures of first trimester microparticle data;
iii) a dataset comprising measures of second trimester microparticle data; and
iv) a comparand output dataset that includes first trimester data compared to second trimester data;
wherein the first trimester microparticle data and the second trimester microparticle data is received by:
I) providing a microparticle-enriched blood sample from a pregnant female subject; and
II) determining a measure of each of a plurality of proteins from the sample;
b) producing a meta-dataset that comprises measures of the plurality of features; and
c) executing by computer a model that infers, from measures of the one or more features in the meta-dataset, a risk of an adverse gestational outcome in the subject.

34. The method of claim 33, wherein the model is a model created by a method of claim 1.

35. A method of treating a pregnant subject comprising:
(I) during pregnancy:
(A) inferring a gestational outcome in a pregnant subject by executing a model on a first meta-dataset that includes for the subject, measures of a plurality of diagnostic features from:
i) one or more first datasets comprising measures of clinical data, wherein the clinical data comprises maternal data inputs and/or conception data inputs; and
(B) based on an inference of low, average or high risk of an adverse gestational outcome, tracking the subject into one of three treatment tracks selected from: traditional prenatal care, prenatal care with telemedicine, and enhanced at risk care;
(II) during the first trimester of pregnancy, and after (I):
(A) inferring a gestational outcome in the subject by executing a model on a second meta-dataset that includes for the subject, measures of a plurality of diagnostic features from:
i) one or more first datasets comprising measures of clinical data, wherein the clinical data comprises maternal data inputs and/or conception data inputs; and
ii) a dataset comprising measures of first trimester microparticle data;
iii) optionally, a first trimester clinical dataset comprising measures of clinical data collected during the first trimester; and
(B) based on an inference of low, average, or high risk of an adverse gestational outcome, tracking the subject into one of three treatment tracks selected from: traditional prenatal care, prenatal care with telemedicine and enhanced at risk care; and
(III) during the second trimester of pregnancy:
(A) inferring a gestational outcome in the subject by executing a model on a third meta-dataset that includes for the subject, measures of a plurality of diagnostic features from:
i) one or more first datasets comprising measures of clinical data, wherein the clinical data comprises maternal data inputs and/or conception data inputs; and
ii) a dataset comprising measures of first trimester microparticle data;
iii) optionally, a first trimester clinical dataset comprising measures of clinical data collected during the first trimester; and
iv) a dataset comprising measures of second trimester microparticle data;
v) a comparand output dataset comparing data from first trimester microparticle data to second trimester microparticle data;
vi) optionally, one or more third clinical datasets comprising measures of clinical data collected during the second trimester; and
(B) based on an inference of low, average, or high risk of an adverse gestational outcome, tracking the subject into one of three treatment tracks selected from: traditional prenatal care, prenatal care with telemedicine, and enhanced at-risk care.

36. The method of claim 35, wherein tracking the subject into one of the three treatment tracks comprise tracking via a model.

37. The method of claim 35, wherein enhanced at-risk care comprise one or more of:
1. Referral to Preterm Birth Prevention Clinic;
2. Referral to Maternal Fetal Medicine specialist;
3. Education on signs/symptoms of preterm labor;
4. Evaluation of medical (i.e. progestogen supplementation, low-dose aspirin) or surgical (i.e. cervical cerclage) options;
5. Modification of behaviors, lifestyle and diet to support a healthy birth outcome;
6. Increased office visits and modified content of office visits;
7. Increased surveillance via ultrasound and cervical length measurements; and
8. Preparation for acute-stage events (i.e. planning for NICU access, education on medicines that can be given upon initiation of preterm labor to extend gestation, mature the baby's lungs, and provide neuroprotective agents for the baby's brain development)

38. A method for treating a pregnant subject at high risk of an adverse gestational outcome comprising:
(A) receiving a meta-dataset that includes for the subject, measures of a plurality of diagnostic features from:
i) one or more first datasets comprising measures of clinical data, wherein the clinical data comprises maternal data inputs and/or conception data inputs; and
ii) a dataset comprising measures of first trimester microparticle data;
iii) optionally, a first trimester clinical dataset comprising measures of clinical data collected during the first trimester; and
iv) a dataset comprising measures of second trimester microparticle data;
v) an output dataset comparing data from first trimester microparticle data to second trimester microparticle data;
vi) optionally, one or more third clinical datasets comprising measures of clinical data collected during the second trimester; and
(B) executing, by computer, a model that, based on measures of the diagnostic features, predicts an adverse gestational outcome in the subject; and
(C) based on an inference of low, average, or high risk of an adverse gestational outcome, tracking the subject into one of three treatment tracks selected from: traditional prenatal care, prenatal care with telemedicine and enhanced at risk care.

39. A system for inferring risk of an adverse gestational outcome comprising:
a controller; and
a database communicatively coupled to the controller, the database comprising:
(1) a meta-dataset comprising measures of features in feature data, wherein the feature data comprises:
i) one or more first datasets comprising measures of clinical data, wherein the clinical data comprises maternal data inputs and/or conception data inputs; and
ii) a dataset comprising measures of first trimester microparticle data;
iii) optionally, a first trimester clinical dataset comprising measures of clinical data collected during the first trimester; and
iv) a dataset comprising measures of second trimester microparticle data;
v) an output dataset comparing data from first trimester microparticle data to second trimester microparticle data;
vi) optionally, one or more third clinical datasets comprising measures of clinical data collected during the second trimester;
(2) a classification model which, based on the measures, infers a gestational outcome in the subject; and
(3) computer executable instructions for implementing the classification model on the meta-dataset.

40. A non-transitory computer readable medium comprising machine executable code, which, when executed by a computer processor, infers an adverse gestational outcome in a subject by:
(a) accessing a meta-dataset measures of features in feature data, wherein the feature data comprises
i) one or more first datasets comprising measures of clinical data, wherein the clinical data comprises maternal data inputs and/or conception data inputs; and
ii) a dataset comprising measures of first trimester microparticle data;
iii) optionally, a first trimester clinical dataset comprising measures of clinical data collected during the first trimester; and
iv) a dataset comprising measures of second trimester microparticle data;
v) an output dataset comparing data from first trimester microparticle data to second trimester microparticle data;
vi) optionally, one or more third clinical datasets comprising measures of clinical data collected during the second trimester; and
(b) executing a computer model on the meta-dataset set to infer an adverse gestational outcome in the subject.

41. A method of treating adverse gestational outcome in a subject comprising:
(a) inferring the presence of adverse gestational outcome in a subject according to a method as described herein; and
(b) administering a therapeutic intervention to the subject effective to treat the adverse gestational outcome.

42. A method for diagnosing and treating an adverse gestational outcome in a subject, the method comprising:
(a) receiving from a subject: (i) pre-pregnancy maternal data, (ii) conception data, (iii) pregnancy maternal data;
(b) receiving from the subject a blood sample;
(c) determining, from a microparticle-enriched portion of the blood sample, data comprising measures of protein features;
(d) generating a meta-dataset for the subject based upon the data;
(e) generating an inference of the adverse gestational outcome in the subject upon processing the meta-dataset with an inference model derived from a population of subjects; and
(f) at an output device associated with the subject, providing a therapy to the subject for the adverse gestational outcome upon processing the inference with a therapy model designed to treat the adverse gestational outcome.

43. A method for creating a model that infers a gestational outcome in a subject in a first trimester of pregnancy comprising:
a) receiving, at a database, a plurality of datasets comprising data on each of a plurality of subjects, wherein the datasets include:
i) one or more datasets comprising measures of clinical data; and
ii) a dataset comprising measures of first trimester microparticle data;
and wherein each dataset includes a gestational outcome identifier for each subject;
b) performing, via a controller, an analysis on each of the datasets, wherein the analyses identify, from each dataset, one or a plurality of dataset features that infer a gestational outcome in subject;
c) receiving, at the database, a meta-dataset that includes, for each subject, measures of a plurality of the identified features from each of the datasets and the gestational outcome identifier; and
d) performing, via the controller, an analysis on the meta-dataset, wherein the analysis produces, from the identified features, a model that infers a gestational outcome for a subject.

44. A method for creating a model that infers a gestational outcome in a subject in post-conception comprising:
a) receiving, at a database, a plurality of datasets comprising data on each of a plurality of subjects, wherein the datasets include:
i) a dataset comprising measures of pre-pregnancy maternal data; and
ii) a dataset comprising measures of conception status data;
and wherein each dataset includes a gestational outcome identifier for each subject;
b) performing, via a controller, an analysis on each of the datasets, wherein the analyses identify, from each dataset, one or a plurality of dataset features that infer a gestational outcome in subject;
c) receiving, at the database, a meta-dataset that includes, for each subject, measures of a plurality of the identified features from each of the datasets and the gestational outcome identifier; and
d) performing, via the controller, an analysis on the meta-dataset, wherein the analysis produces, from the identified features, a model that infers a gestational outcome for a subject.
